(19) United States
(12) Patent Application Publication
(10) Pub. No.: US 2005/0112216 A1
(43) Pub. Date: May 26, 2005

(54) Whole grain corn alone levels blood sugar

(76) Inventor: Dwight Reibling, Sturgis, MI (US)

Correspondence Address: Dwight Reibling, 25941 Antler, Sturgis, MI, 49091, US

(21) Appl. No.: 10/719,273

(22) Filed: Nov. 20, 2003

(51) Int. Cl. A61K 35/78

(52) U.S. Cl. 424/750

(57) ABSTRACT

A novel use of whole grain corn alone, to level blood sugar is what this invention is all about. 30-35 grams, eaten in 5 gram portions, 6-7 times a day, about 3 hour intervals, the last one before going to bed is the day's therapy. Whole grain corn is modified for good human consumption. This invention uses 2. 1. soaked in water until chewable. 2. grinding into 3 distinct fineness, fine, medium fine and coarse 2 millimeters in diameter. Diabetics using the products of this invention have a 24 hour safety net guarding against too high or too low sugar level. Sweeteners and flavoring can be used in the processed product, but never compromise the purity and integrity of the invention; whole grain corn alone levels blood sugar.

### BACKGROUND—DESCRIPTION OF PRIOR ART

It is generally established there are 17 million American diabetics. My wife Marion is one. In the “Saturday Evening Post” September-October 2003 issue reports that one out of every 3 births in year 2000 will become diabetic. (Page 40, second paragraph). The article further reports, these 17 million will be added to the already 17 million.

The disease is horrific! It is distressful to live with, its complication worse. Science and medicine are doing their best to find a cure. In the meantime, we treat the symptoms.

There is a wealth of medical and scientific knowledge available to a few. Very little of this overflows into the general public knowledge.

My invention comes more from the general public knowledge. My invention is, whole grain corn alone levels blood sugar. The major source of my knowledge comes from the pool of “general public information and knowledge.”

In hand is a printout of U.S. Pat. No. 6,339,076, issued to Kaufman, Jan. 15, 2002. Having read and studied the overwhelming medical, scientific and clinical test of this patent, I asked myself the question, “How can a person with public knowledge ever access his own invention over against that?”

I also have a printout (5 pages), same patent numbers, same inventions, Kaufman, dated Aug. 14, 1998. The inventor abstracts the invention, details the invention and cites the use of uncooked corn starch 7 times as the main complex carbohydrate ingredient.

The inventor then adds many, many other ingredients; simple sugars, nuts, and oils found in most candy bars at the candy counter. If uncooked cornstarch is the main complex carbohydrate, why so little of it? The inventor compromised their own objective in the product. Every candy bar at the candy counter that has a little cornstarch is the same in essential content.

On Jul. 15, 2003 my wife ordered by mail two, 4 bar packages of the invention. I have the box before me and read the following nutrition facts, some, but not all:
40 grams per serving bar
31 grams of carbohydrate of which 10 grams are sugar
5 grams sugar alcohol

Below the nutrition facts, I see where uncooked corn starch is fifth in rank. The question remains, how much of the essential complex carbohydrate uncooked corn starch is in the invention and product of the invention?

I have under investigation a printout of U.S. Pat. No. 6,156,738, having an appeal number 241004, filed Feb. 1, 1999, by Bell, et al. I also have a printout of patent issued to Bell, et al, U.S. Pat. No. 6,156,737 with a date of Dec. 5, 2000.

I give honor to the above inventors for their medical and scientific knowledge and skill. How much distinguishable difference is there between the above two inventions? I see a parallel similarity.

Looking at claims 4, 12, 21 and 24 there are 25 ingredients; 3 simple carbohydrates, 12 complex, 7 protein and 3 fat (oils). When all 25 ingredients are put together in a product, how much uncooked corn starch is in the product?

A diabetic with public knowledge and skill can achieve the same results with a handful of chocolate covered peanuts and a full glass of whole milk before bedtime and for less money too!

This evaluation may be harsh and unkind but necessary. The inventors' goal is compromised by their own knowledge and skill. Betrayed by their many claims.

My invention is “Whole grain corn alone levels blood sugar.” Let's take a look at what whole grain corn alone provides as compared with a little uncooked corn starch and many, many other ingredients.

| | | Whole | Corn |
|---|---|---|---|
| | | Corn/Flour | Starch |
| | | 1 Cup | 1 Cup |
| Weight | g | 117 | 128 |
| Calories | | 422 | 488 |
| Carbohydrates | g | 89.9 | 116.8 |
| Cholesterol | mg | 0 | 0 |
| Dietary fiber | g | 15.7 | 1.2 |
| Total fat | g | 4.5 | 0 |
| Mono fat | g | 1.2 | 0 |
| Poly fat | g | 2.1 | 0 |
| Sat fat | g | 0.6 | 0 |
| Protein | g | 8.1 | 0.4 |
| Calcium | mg | 8 | 4 |
| Iron | mg | 3 | 0 |
| Potassium | mg | 369 | 4 |
| Sodium | mg | 6 | 12 |
| Zinc | mg | 2 | 0 |
| Vitamin A | RE | 56 | 0 |
| Vitamin B12 | mcg | 0 | 0 |
| Vitamin B6 | mg | 0.43 | 0 |
| Vitamin C | mg | 0 | 0 |
| Folic acid | mcg | 29.3 | 0 |
| Niacin | mg | 2.2 | 0 |
| Riboflavin | mg | .09 | 0 |
| Thiamin | mg | .29 | 0 |

Compared to the cited patents, my invention has in whole grain corn alone what they never achieved with their many, many ingredients. They compromised their uncooked corn starch, while whole grain corn alone has first and foremost corn starch, plus protein (about 8%) and oil, without all of their candy bar stuff.

Dr. Mette Axelson, Lundberg Laboratory for Diabetes Research, Institute of Internal medicine, Sahlgrenska University Hospital, S-413 45 Göteberg, Sweden. Reports in his abstract:
1) 30 grams of uncooked corn starch before bedtime reduced by 70% nocturnal hypoglycemia in patients having Type I diabetes.
2) Ingestion of uncooked corn starch also has the leveling of blood sugar for Type II diabetes, long before macroangropathy sets in.

I am grateful for this study for it confirms the value of uncooked corn starch. Better still is the outstanding value of whole grain corn alone levels blood sugar.

### SUMMARY OBJECT AND ADVANTAGES

Accordingly, besides the objective and advantages of “whole grain corn” invention are as follows:
1. to provide in a single simple complex carbohydrate all that is necessary to achieve level blood sugar.
2. to provide a product in many convenient forms.
3. To provide a product that gives 24 hour therapy in level blood sugar.
4. To be cost effective.
5. To be available everywhere a person shops.
   Operation:

Uncooked corn starch has 7-9 hours of absorption rate. Modified whole grain corn with one-third of itself course ground (2-millimeters in diameter) lengthens absorption rate up to 7-12 hours. The absorption rate of whole grain corn parallels the 24 hours activity profile of Lantus insulin.

There are 2 models of modified corn in said abstract of invention.

Model 1—after whole grain corn is softened by water is chewable, even with dentures. Taste can be enhanced by adding flavoring and sweeteners; but not simple sugars.

Model 2—lends itself to many edible forms; a tablespoon of said modified whole grain corn:
1. a tablespoon of whole ground corn alone.
2. a tablespoon of said corn mixed in a glass of water, milk, cup of coffee, 4 oz. yogurt, unsweetened applesauce, etc.
3. a tablespoon of said corn mixed with peanut butter, a spread or as is.
4. a tablespoon of said corn can be put in a small paper like packet for convenient use.
5. said model 2 can be processed with a tablespoon of said corn into a compressed tablet form.

Models 1 and 2 can be enhanced in taste by the use of sweeteners and flavoring; but in no case shall simple sugars be used. The integrity of “whole grain corn alone levels blood sugar” is the main thrust of the invention.

The invention is broad in its design use, and narrow in its outcome.

The invention calls for 25-30 grams of whole grain corn consumption in one day. The design further calls for 6-7 consumption times of 5 grams of said modified corn: 6 AM, 9 AM, 12 noon, 3 PM, 6 PM, 9 PM and bedtime.

The said use design with said absorption rate provides a safe, all the time, blood sugar level.

The said design use achieves level blood sugar where one night time use in other inventions fall far short of the ultimate goal.

Further consideration of objective, operation and advantages is that of convenience of use, cost, and easy to find. Both said models of modified whole grain corn is as use convenient as a pint jar of softened corn setting on the kitchen counter; or as use convenient as a box of said ground corn, a tablespoon measure, and a glass of water.

The compressed tablet or paper packet of said product extends convenience of use beyond the home.

Oh, yes, what about cost and easy to find in the market.

Said model 1 is as cost effective as ajar or pickles and would represent about one month's consumption.

Said model 2 about the same cost as a 5 pound package of all purpose wheat flour.

How easy to find? I searched in four major national markets, inquired about therapeutic diabetic blood products and found not one. One comment by a druggist was to the effect it cost too much to inventory with few sales.

My invention is affordable for every diabetic, and will be as easy to find as Kellogg Corn Flakes everywhere you shop.

1. Modified whole grain corn alone is blood level controlling.

2. Said modified whole grain corn has two models.

3. Said model number 1 is whole grain corn softened in water.

4. Said model number 2 is whole grain corn ground into 3 definable fineness; fine, medium fine and course; being 2 millimeters in diameter.

5. The modified whole grain corn in claims 1, 2, 3 and 4 is processed in temperatures less than 160° F.

6. The modified whole grain corn in claims 3 and 4 have a design use to be followed.

7. The quantity of modified whole grain corn in said design use is 5 grams of carbohydrates.

8. Said 5 grams quantity in claim 7 is used: 6 AM, 9 AM, 12 noon, 3 PM, 6 PM, and at bedtime.

9. The total consumption in said claim 8 is 30 grams of carbohydrates in 24 hours.
